(19) United States
(12) Patent Application Publication
(10) Pub. No.: US 2023/0321287 A1
(43) Pub. Date: Oct. 12, 2023

(54) RADIOLABELLED GRPR-ANTAGONIST FOR USE AS THERAGNOSTIC

(71) Applicant: NOVARTIS AG, Basel (CH)

(72) Inventors: Daniela CHICCO, Bollengo (IT); Maurizio F. MARIANI, Ivrea (IT); Francesca ORLANDI, Ivrea (IT); Antje WEGENER, Saint nom la Breteche (FR)

(21) Appl. No.: 17/754,059

(22) PCT Filed: Sep. 23, 2020
(86) PCT No.: PCT/EP2020/076542

§ 371 Date: Mar. 22, 2022

(30) Foreign Application Priority Data
Sep. 24, 2019 (EP) 19199169.4
Jul. 2, 2020 (EP) 20183788.7

(51) Int. Cl. A61K 51/08; A61P 35/00

(52) U.S. Cl. CPC A61K 51/088; A61P 35/00

(57) ABSTRACT

The present disclosure relates to gastrin-releasing peptide receptor (GRPR) targeting pharmaceuticals and their use in a theragnostic approach for selection and therapy of subjects with GRPR-expressing malignancies. In particular, the present disclosure relates to a pharmaceutical composition of a radiolabeled GRPR-antagonist, for use in treating GRPR-positive tumors in a human subject selected for said treatment, wherein said subject has been selected for the treatment by PET/CT or PET/MRI imaging with a corresponding ⁶⁸Ga-labelled GRPR antagonist as contrast agent.

### TECHNICAL FIELD

The present disclosure relates to gastrin-releasing peptide receptor (GRPR) targeting radiopharmaceuticals and their use in a theragnostic approach for selection and therapy of subjects with GRPR-expressing malignancies. In particular, the present disclosure relates to a pharmaceutical composition of a radiolabeled GRPR-antagonist, for use in treating GRPR-positive tumors in a human subject eligible for said treatment, wherein said subject has been selected for the treatment by PET/CT or PET/MRI imaging with the same GRPR antagonist but with 68-Ga as radiometal for use as contrast agent.

### BACKGROUND ART

Bombesin was first isolated from the European frog Bombina bombina and was demonstrated to mimic the mammalian gastrin-releasing peptide (GRP) and neuromedin B (NMB): Erspamer, V. Discovery, Isolation, and Characterization of Bombesin-like Peptides. Ann N Y Acad Sci 547: 3-9, 1988 ; Jensen, R.T.; Battey, J.F.; Spindel, E.R.; Benya, R.V. International union of pharmacology. LXVIII. Mammalian bombesin receptors: Nomenclature, distribution, pharmacology, signaling, and functions in normal and disease states. Pharmacol. Rev. 2008, 60, 1-42].

Gastrin-releasing peptide (GRP), a bombesin-like peptide growth factor, regulates numerous functions of the gastrointestinal and central nervous systems, including release of gastrointestinal hormones, smooth muscle cell contraction, and epithelial cell proliferation. It is a potent mitogen for physiologic and neoplastic tissues, and it may be involved in growth dysregulation and carcinogenesis.

The effects of GRP are primarily mediated through binding to its receptor, the GRP receptor (GRPR), a G protein-coupled receptor originally isolated from a small cell lung cancer cell line. Upregulation of the pathway of GRP/GRPR has been reported in several cancers, including breast, prostate, uterus, ovaries, colon, pancreas, stomach, lung (small and non-small cell), head and neck squamous cell cancer and in various cerebral and neural tumours.

In breast cancer, GRPR overexpression can reach very high density according to tumour type (e.g. 70-90% expression in ductal breast cancer specimens) [Van de Wiele C, et al. J Nucl Med 2001, 42(11):1722-1727].

GRPR are highly overexpressed in prostate cancer where studies in human prostate cancer cell-lines and xenograft models showed both high affinity (nM level) and high tumour uptake (%ID/g) but the relative expression of GRPR across evolving disease setting from early to late stage has not been fully elucidated yet [Waters, et al. 2003, Br J Cancer. Jun 2; 88(11): 1808-1816].

In colorectal patients, presence of GRP and expression of GRPR have been determined by immunohistochemistry in randomly selected colon cancers samples, including LN and metastatic lesions. Over 80% of samples aberrantly expressed either GRP or GRPR, and over 60% expressing both GRP and GRPR, whereas expression was not observed in adjacent normal healthy epithelium [Scopinaro F, et al. Cancer Biother Radiopharm 2002, 17(3):327-335].

GRP is physiologically present in pulmonary neuroendocrine cells and plays a role in stimulating lung development and maturation. However, it seems to also be involved in growth dysregulation and carcinogenesis. Stimulation of GRP leads to increasing the release of epidermal growth factor receptor (EGFR) ligands with subsequent activation of EGFR and mitogen-activated protein kinase downstream pathways. Using non-small cell lung cancer (NSCLC) cell lines it has been confirmed that EGF and GRP both stimulate NSCLC proliferation, and inhibition of either EGFR or GRPR resulted in cell death [Shariati F, et al. Nucl Med Commun 2014, 35(6):620-625].

In nuclear medicine, peptide receptor agonists have long been the ligands of choice for tracer development and utilization. The rationale behind the use of agonist-based constructs laid on to receptor-radioligand complex internalization enabling the high accumulation of radioactivity inside the target cells. In case of radiometal-labelled peptides, the efficient receptor-mediated endocytosis in response to agonist stimulation provides high in vivo radioactivity uptake in targeted tissues, a crucial prerequisite for optimal imaging of malignancies. However, a paradigm shift occurred when receptor-selective peptide antagonists showed preferable biodistribution, including considerably greater in vivo tumour uptake, compared with highly potent agonists. A further advantage displayed by GRPR antagonists is a safer clinical use, not so much at tracer doses for the current diagnostic point of view, but in view of greater doses for potential therapeutic purposes, as the use of antagonists does not foresee acute biological adverse effects [Stoykow C, et al. Theragnostics 2016, 6(10):1641-1650].

In non-clinical models, [68Ga]-NeoB and [177Lu]-NeoB (also called [68Ga]-NeoBOMB1 and [177Lu]-NeoBOMB1) have shown high affinity to the GRPR expressed in breast, prostate, and Gastro Intestinal Stromal Tumor (GIST), as well as a low degree of internalization upon binding to the specific receptor. The ability of the radiolabeled peptide to target the GRPR expressing tumor has been confirmed in in vivo imaging and biodistribution studies in animal models [Dalm et al Journal of nuclear medicine 2017, Vol. 58(2) : 293-299; Kaloudi et al. Molecules, 2017 Nov 11;22(11); Paulmichl A et al. Cancer Biother Radiopharm, 2016 Oct;31(8):302-310].

Despite many therapeutic advances, a number of common tumors such as cancer of the breast, prostate, GIST, Head and Neck and CNS, are still a frequent cause of death and new treatment approaches are needed.

In this context, it would thus be desirable to provide a novel theragnostic approach for selection and therapy of GRPR-expressing malignancies.

The present disclosure relates to a theragnostic approach based on the use of a radiolabeled GRPR antagonist with (1) Gallium 68 (68Ga) to identify tumor lesions and (2) Lutetium-177 (177Lu) for the treatment of these tumor lesions, in particular on breast, prostate, lung (small cell and non-small cell) colon-rectum. GIST, neuroblastoma, glioblastoma and renal.

### SUMMARY

The present disclosure relates to a pharmaceutical composition of a radiolabeled GRPR-antagonist, for use in treating GRPR-positive tumors in a human subject selected for said treatment, wherein said pharmaceutical composition comprises
a radiolabeled GRPR-antagonist of the following formula:

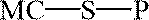

wherein:
   M is a radiometal suitable for therapy, typically 177-Lutetium, and C is a chelator which binds M; e.g. by forming a complex with M;
   S is an optional spacer covalently linked between C and the N-terminal of P;
   P is a GRP receptor peptide antagonist covalently bound with its N-terminal to C or to S; typically of the general formula : 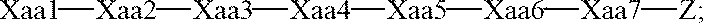
   Xaa1 is not present or is selected from the group consisting of amino acid residues Asn, Thr, Phe, 3- (2-thienyl) alanine (Thi), 4-chlorophenylalanine (Cpa, α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
   Xaa2 is Gln, Asn or His;
   Xaa3 is Trp or 1, 2, 3, 4-tetrahydronorharman-3-carboxylic acid (Tpi);
   Xaa4 is Ala, Ser or Val;
   Xaa5 is Val, Ser or Thr;
   Xaa6 is Gly, sarcosine (Sar), D-Ala, or β-Ala;
   Xaa7 is His or (3-methyl )histidine (3-Me)His;
   (all amino acids being as L- or D-isomers)
   Z is selected from -NHOH, -NHNH2, -NH-alkyl, -N(alkyl)2, and -O-alkyl or Z is 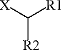
   wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido or amide substituted aryl or heteroaryl group; and,
one or more pharmaceutically acceptable excipients,
wherein said subject has been selected for the treatment, by positron emitting tomography (PET) / computed tomography (CT) or PET/ magnetic resonance imaging MRI with the same GRPR antagonist as defined for the treatment but with ⁶⁸Ga as radiometal for use as contrast agent.

Similarly the disclosure relates to a radiolabeled GRPR-antagonist for use in the preparation of a pharmaceutical composition for treating GRPR-positive tumors in a human subject, wherein said pharmaceutical composition comprises
a radiolabeled GRPR-antagonist of the following formula:

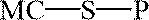

wherein:
   M is a radiometal suitable for therapy, typically 177-Lutetium, and C is a chelator which binds M; e.g. by forming a complex with M;
   S is an optional spacer covalently linked between C and the N-terminal of P;
   P is a GRP receptor peptide antagonist covalently bound with its N-terminal to C or to S; typically
wherein said subject has been selected for the treatment, by positron emitting tomography (PET) / computed tomography (CT) or PET/ magnetic resonance imaging MRI with the same GRPR antagonist as defined for the treatment but with ⁶⁸Ga as radiometal for use as contrast agent.

The disclosure also relates to the pharmaceutical composition of a radiolabeled GRPR-antagonist, for use as a contrast agent for PET/CT or PET/MRI imaging in determining whether a subject can be selected for a treatement with radiolabelled GRPR antagonist for treating GRPR-positive tumors.

In specific embodiments, P is of the general formula:

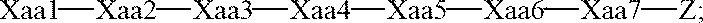

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
(all amino acids being as L- or D-isomers)
Z is selected from —NHOH, —NHNH2, -NH-alkyl, -N(alkyl)2, and -O-alkyl or Z is

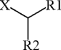

wherein X is NH (amide) or O (ester) and R1 and R2 are the same or different selected from a proton, an optionally substituted alkyl, an optionally substituted alkyl ether, an aryl, an aryl ether or an alkyl-, halogen, hydroxyl, hydroxyalkyl, amine, amino, amido or amide substituted aryl or heteroaryl group; and,
one or more pharmaceutically acceptable excipients.

In specific embodiments, P is DPhe-Gln-Trp-Ala-Val-Gly-His- NH-CH(CH₂-CH(CH₃)₂)_{2.}

In particularly preferred embodiments, the radiolabelled GRPR-antagonist for use as a therapeutic agent is M-NeoB of formula (I):

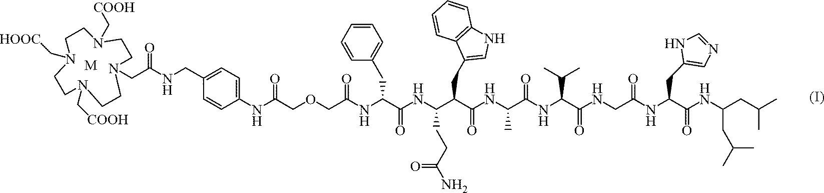

wherein M is a ¹⁷⁷Lu.

In preferred embodiments, the pharmaceutical composition for use as contrast agent comprises a radiolabelled GRPR-antagonist M-NeoB of formula (I):

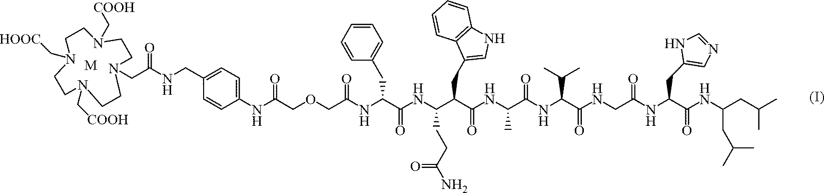

wherein M is a ⁶⁸Ga.

In specific embodiments, a therapeutically efficient dose amount of radiolabeled GRPR-antagonist administered to the subject ranges from 1.85 to 18.5 GBq (50-500 mCi) in 1-8 cycles of infusion.

In specific embodiments, the subject has been selected for the treatment by evaluating [⁶⁸Ga]-labeled GRPR antagonist uptake in the lesions as determined by PET/MRI or PET/CT imaging in said subject.

For example, a subject is selected for the treatment if said subject fulfils the following condition: at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging in said subject.

In specific embodiment, said subject has GRPR-positive solid tumors selected among gastrointestinal stromal tumor (GIST), neuroblastoma, glioblastoma, breast, prostate, lung (small cell and non-small cell), colon-rectum, and renal cancer, preferably breast cancer.

In specific embodiments, an imaging efficient dose amount of radiolabeled GRPR-antagonist administered to the patient ranges from 150-250 MBq.

The disclosure also relates to a method for determining whether a human patient having tumors can be selected for a treatment with a radiolabelled GRPR antagonist, said method comprising the steps of:
(i) administering an efficient amount of a radiolabelled GRPR antagonist as a contrast agent for imaging the uptake of said radiolabelled GRPR antagonist,
(ii) acquiring an image by PET/MRI or PET/CT of said patient, and
(iii) comparing with a control image.

In specific embodiments, the above method further comprises a step of treating GRPR-positive cancer by administering a therapeutically efficient amount of a therapeutic agent which comprises the same GRPR antagonist used in step (i) but having a radiometal suitable for therapy, for example ¹⁷⁷Lu.

In specific embodiments of the above method, the therapeutic agent is administered at least two weeks after step (i).

### DETAILED DESCRIPTION

The disclosure relates to a pharmaceutical composition of a radiolabeled gastrin-releasing peptide receptor (GRPR)-antagonist, for use in treating GRPR-positive tumors in a human subject, wherein said pharmaceutical composition comprises
a radiolabeled GRPR-antagonist of the following formula:

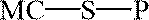

wherein:
   M is a radiometal suitable for therapy, typically 177-Lutetium, and C is a chelator which binds M;
   S is an optional spacer covalently linked between C and the N-terminal of P;
   P is a GRP receptor peptide antagonist covalently bound with its N-terminal to C
   or to S; and,
one or more pharmaceutical excipients,
wherein said subject has been selected for the treatment by positron emitting tomography (PET) / computed tomography (CT) or PET/ magnetic resonance imaging MRI with the same GRPR antagonist as defined for the treatment but with ⁶⁸Ga as radiometal for use as contrast agent.

### Definitions

The phrase “treatment of” and “treating” includes the amelioration or cessation of a disease, disorder, or a symptom thereof. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease.

As used herein, the terms “substituted” or “optionally substituted” refers to a group which is optionally substituted with one or more substituents selected from: halogen, —OR′, —NR′R″, —SR′, —SiR′R″R′”, —OC(O)R′, —C(O)R′, —CO₂R′, —C(O)NR′R″, —OC(O)NR′R″, —NR″C(O)R′, —NR′—C(O)NR″R″’, —NR″C(O)OR′, —NR—C(NR′R″R″′)═NR⁗, —NR—C(NR′R″)═NR′″—S(O)R′, —S(O)₂R′, —S(O)₂NR′R″, —NRSO₂R’, —CN, —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R‴ and R⁗ may be independently selected from hydrogen, alkyl, heteroalkyl, cycloalkyl, heterocycloalkyl, aryl and heteroaryl. When a compound of the disclosure includes more than one R group, for example, each of the R groups is independently selected as are each R′, R″, R‴ and R⁗ groups when more than one of these groups is present.

As used herein, the terms “alkyl”, by itself or as part of another substituent, refer to a linear or branched alkyl functional group having 1 to 12 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “heteroaryl” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “aryl” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

As used herein, the term “halogen” refers to a fluoro (—F), chloro (—Cl), bromo (—Br), or iodo (—I) group

As used herein the terms “optionally substituted aliphatic chain” refers to an optionally substituted aliphatic chain having 4 to 36 carbon atoms, preferably 12 to 24 carbon atoms.

### The Subject in Need of Radiolabeled GRPR-Antagonist Treatment

The terms “patient” and “subject” which are used interchangeably refer to a human being, including for example a subject that has cancer, more specifically, a patient that has GRPR-positive tumor lesions, as identified for example by ⁶⁸Ga-NeoB PET according to methods described in the examples.

As used herein, the terms “cancer” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. Unless specified otherwise, the term is meant to include all types of cancerous growths or oncogenic processes, metastatic tissues or malignantly transformed cells, tissues, or organs, irrespective of histopathologic type or stage of invasiveness.

In specific embodiments, the cancer is selected from prostate cancer, breast cancer, small cell lung cancer, colon carcinoma, gastrointestinal stromal tumors, gastrinoma, glioma, glioblastoma, renal cell carcinomas, gastroenteropancreatic neuroendocrine tumors, oesophageal squamous cell tumors, neuroblastomas, head and neck squamous cell carcinomas, as well as ovarian, endometrial and pancreatic tumors displaying neoplasia-related vasculature that is GRPR-positive.

In specific embodiments, the cancer is breast, prostate, lung (small cell and non-small cell) colon-rectum GIST, neuroblastoma, glioblastoma or renal cancer. Preferably, the cancer is breast cancer.

### Radiolabeled GRPR-Antagonist for Use According to the Disclosure

The present disclosure relates to a theragnostic approach for treating GRPR-positive tumors in a subject in need thereof.

The theragnostic approach advantageously comprises a first imaging step using a radiolabelled GRPR antagonist for selecting patient with GRPR-positive tumors for the treatment with radiolabelled GRPR-antagonist, and a second treatment step for treating the patient with the corresponding radiolabelled GRPR-antagonist.

Hence, in specific embodiments, the same GRPR-antagonist compound is used for the imaging step for selecting the patient for the treatment and for the treatment step, but the radiometal is different, one being suitable for use as contrast agent for imaging, and the other for use as therapeutic agent for nuclear therapy.

As used herein, the GRPR-antagonist has the following formula:

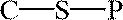

wherein:
C is a chelator which has the capacity to bind a radiometal M;
S is an optional spacer covalently linked between C and the N-terminal of P;
P is a GRP receptor peptide antagonist.
GRP receptor peptide antagonists have been described in the art and include derivatives of the bombesin (BBN) agonist peptide (Pyr-Gln-Arg-Leu-Gly-Asn-Gln-Trp-Ala-Val-Gly-His-Leu-Met-NH2). The minimum amino acid sequence required for GRPR binding has been indicated as BBN(7-14). Derivatives of BBN(7-14) with antagonist properties have then been developed, in particular with modifications or deletions of the amino acids 13 and 14. Examples of GRPR antagonists include RM2, SB3, NeoB (also called NeoBOMB1), RM26, BAY864367, CB-TE2A-AR06 and ProBOMB1 as described respectively in Mansi, et al. J. Nucl. Med. 2016, 57, 67S-72S. Sah, et al. J. Nucl. Med. 2015, 56, 372-378. Zang, et al. Clin. Nucl. Med. 2018, 43, 663-669. Nock, B. et al.. J. Nucl. Med. 2016, 58, 75-80. Maina, T.; et al. Eur. J. Nucl. Med. Mol. Imaging 2015, 43, 964-973. Kahkonen, et al. Clin. Cancer Res. 2013, 19, 5434-5443. Wieser, G.; et al. Theranostics 2014, 4, 412-419.

The chemical structure of ProBOMB1, a derivative of bombesin, is disclosed hereafter:

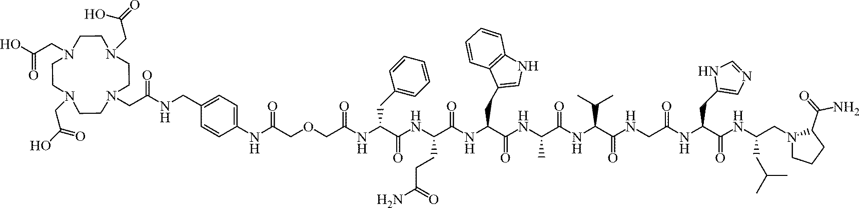

In specific embodiments, P is a GRP receptor peptide antagonist of the general formula :

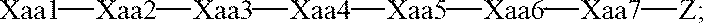

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
all amino acids being, independently, D- or L- isomers,
Z is selected from —NHOH, —NHNH2, -NH-alkyl, -N(alkyl)2, and —O—alkyl or Z is

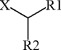

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group.;

According to an embodiment, Z is selected from one of the following formulae, wherein X is NH or O:

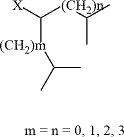

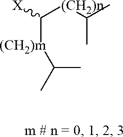

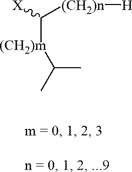

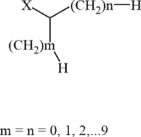

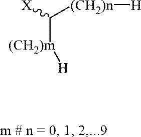

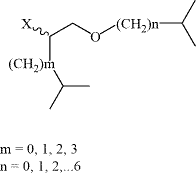

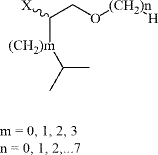

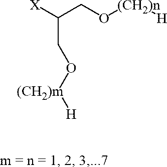

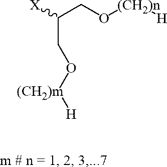

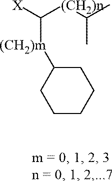

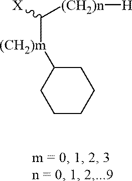

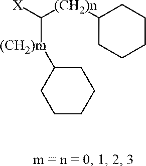

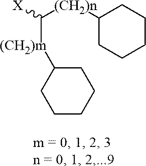

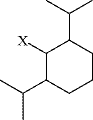

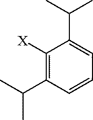

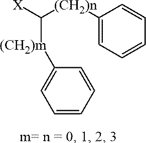

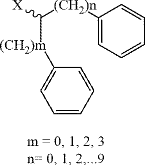

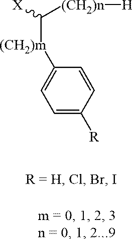

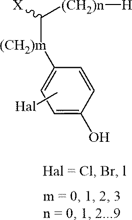

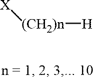

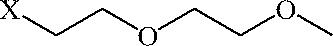

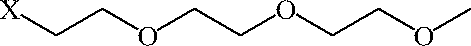

According to an embodiment, P is DPhe-Gln-Trp-Ala-Val-Gly-His-Z; wherein Z is defined as above.

According to an embodiment, P is DPhe-Gln-Trp-Ala-Val-Gly-His-Z;

Z is selected from Leu-ψ(CH2N)-Pro-NH2 and NH—CH(CH₂—CH(CH₃)₂)₂ or Z is

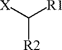

wherein X is NH (amide) and R2 is CH₂—CH(CH₃)₂ and R1 is the same as R2 or is different, for example (CH2N)—Pro—NH2.

According to an embodiment, the chelator C is obtained by grafting one chelating agent selected among the following list:

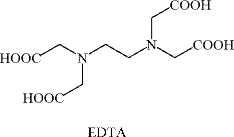

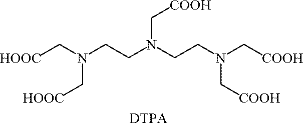

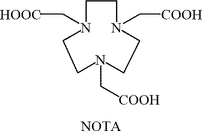 [SMILES: O=C(O)CN1CCN(CC(=O)O)CCN(CC(=O)O)CC1 ; "NOTA"]

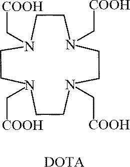

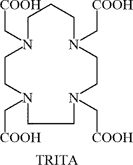

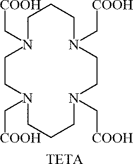

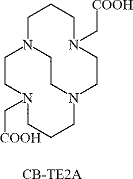

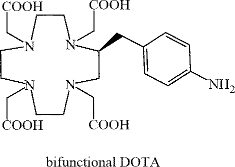 [SMILES: Nc1ccc(C[C@H]2CN(CC(=O)O)CCN(CC(=O)O)CCN(CC(=O)O)CCN2CC(=O)O)cc1 ; "bifunctional DOTA"]

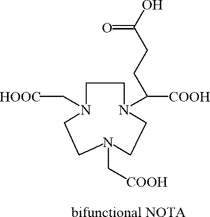 [SMILES: O=C(O)CCC(C(=O)O)N1CCN(CC(=O)O)CCN(CC(=O)O)CC1 ; "bifunctional NOTA"]

In specific embodiments, C is obtained by grafting a chelating agent selected from the group consisting of:

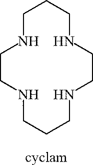

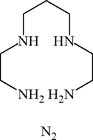 [SMILES: NCCNCCCNCCN ; "N2"]

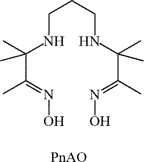

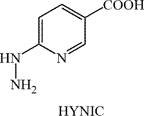

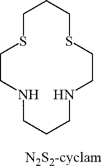

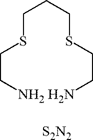 [SMILES: NCCSCCCSCCN ; "S2N2"]

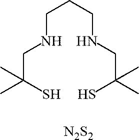

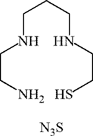

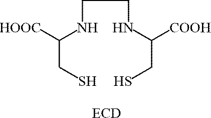

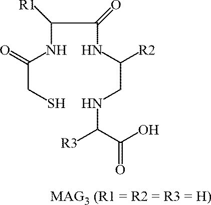

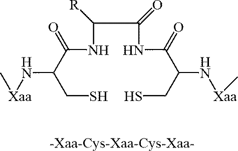

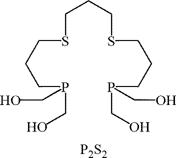

According to an embodiment, the chelator C is selected from the group consisting of DOTA, DTPA, NTA, EDTA, DO3A, NOC and NOTA, preferably is DOTA.

According to an embodiment, S is selected from the group consisting of:
a) aryl containing residues of the formulae:

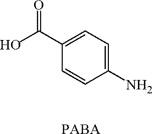

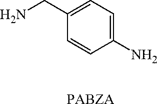

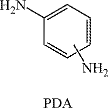

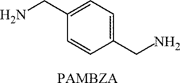

wherein PABA is p-aminobenzoic acid, PABZA is p-aminobenzylamine, PDA is phenylenediamine and PAMBZA is (aminomethyl) benzylamine ;
b) dicarboxylic acids, ω-aminocarboxylic acids,ω-diaminocarboxylic acids or diamines of the formulae:

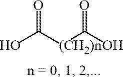

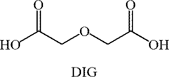

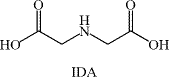

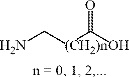

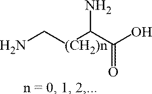

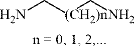

wherein DIG is diglycolic acid and IDA is iminodiacetic acid;
c) PEG spacers of various chain lengths, in particular PEG spacers selected from the following:

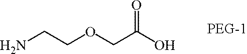

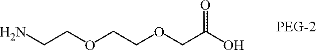 [SMILES: NCCOCCOCC(=O)O]

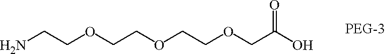 [SMILES: NCCOCCOCCOCC(=O)O]

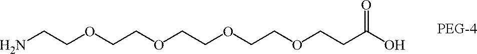 [SMILES: NCCOCCOCCOCCOCCC(=O)O]

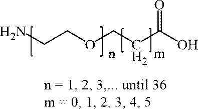

d) α- and β-amino acids, single or in homologous chains various chain lengths or heterologous chains of various chain lengths, in particular:

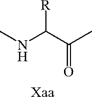

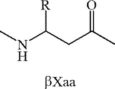

GRP(1-18), GRP(14-18), GRP(13-18), BBN(1-5), or [Tyr4] BB (1-5) ; or
e) combinations of a, b, c and d.

According to a particular embodiment, the radiolabelled GRPR antagonist is selected from the group consisting of compounds of the following formulae:

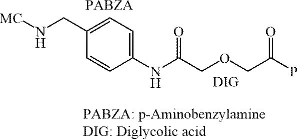

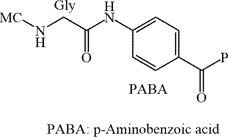

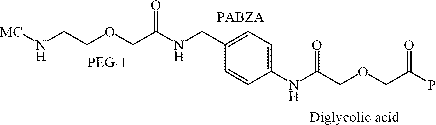

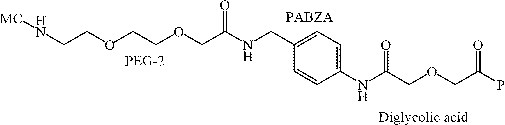

wherein C and P are as defined above, and M is a radiometal.

According to an embodiment P is DPhe-Gln-Trp-Ala-Val-Gly-His-NH-CH(CH₂-CH(CH₃)₂)₂.

According to an embodiment, the GRPR-antagonist is NeoB (also called NeoBOMB1) of formula (II):

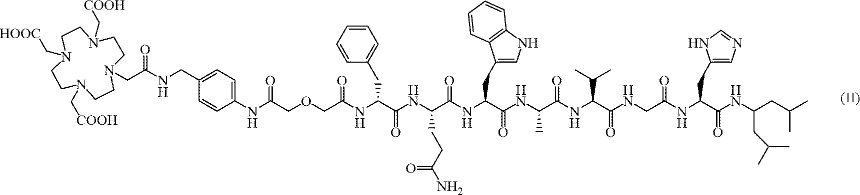 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

DOTA-(p-aminobenzylamine-diglycolic acid)-D-Phe-Gln-Trp-Ala-Val-Gly-His-NH-CH[CH₂-CH(CH₃)₂]₂

According to an embodiment, the radiolabeled GRPR-antagonist is M-NeoB of the following formula (I):

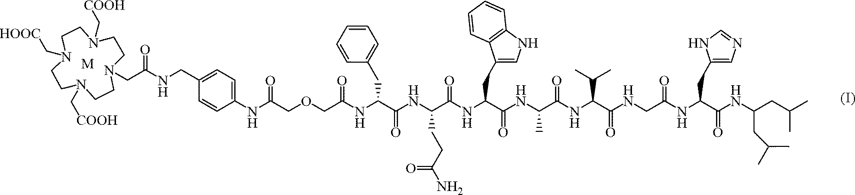

wherein M is radiometal.

According to an embodiment, the radiolabeled GRPR-antagonist is radiolabeled M-NeoBOMB2 of formula (III):

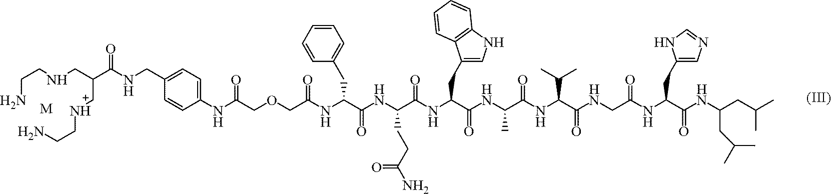

M-N₄ (p-aminobenzylamine-diglycolic acid)-D-Phe-Gln-Trp-Ala-Val-Gly-His-NH-CH[CH₂-CH(CH₃)₂]₂; wherein M is a radiometal.

In an embodiment, M is a radiometal which can be selected from selected from, ¹¹¹In, ^{133m}In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ²¹²Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ²¹²Bi, ¹⁴²Pr, ¹⁴³Pr, ¹⁹⁸Au, ¹⁹⁹Au, ⁸⁹Zr, ²²⁵Ac and ⁴³Sc, ⁴⁴Sc, ⁴⁷Sc. Preferably M is selected from ¹⁷⁷Lu for use in therapy and ⁶⁸Ga for use as contrast agent in imaging.

Typical radiometal suitable for use as contrast agent in PET imaging include the following:

¹¹¹In, ^{133m}In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{117m}Sn, ⁸⁹Zr, ⁴³Sc, ⁴⁴Sc.

According to a preferred embodiment for use in PET imaging, M is ⁶⁸Ga.

A specific embodiment of a radiometal M for use in PET imaging is ⁶⁸Ga. In this case, the radiolabeled GRPR-antagonist can be used as contrast agent for PET/CT or PET/MRI imaging for the patient selection step.

Typical radiometal for use in the treatment step for nuclear medicine therapy include the following: ¹⁶⁹Er, ²¹²Pb, ⁶⁴Cu, ⁶⁷Cu, ¹⁸⁶Re, ¹⁸⁸Re, ⁹⁰Y, ¹⁷⁷Lu, ¹⁶¹Tb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ²¹²Bi, ¹⁴²Pr, ¹⁴³Pr, ¹⁹⁸Au, ¹⁹⁹Au, ²²⁵Ac, ⁴⁷Sc.

According to a preferred embodiment, M is ¹⁷⁷Lu.

### The Pharmaceutical Composition for Use in the Treatment Step

The pharmaceutical composition for use in the treatment step comprises a radiolabeled GRPR-antagonist as described herein and one or more pharmaceutically acceptable excipients.

The radiolabeled GRPR-antagonist can be present in a concentration providing a volumetric radioactivity of at least 100 MBq/mL, preferably at least 250 MBq/mL. The radiolabeled GRPR-antagonist can be present in a concentration providing a volumetric radioactivity comprised between 100 MBq/mL and 1000 MBq/mL, preferably between 250 MBq/mL and 500 MBq/mL, for example, at a concentration of about 370 MBq/mL (10 mCi/mL).

The pharmaceutically acceptable excipient can be any of those conventionally used, and is limited only by physico-chemical considerations, such as solubility and lack of reactivity with the active compound(s).

In particular, the one or more excipient(s) can be selected from stabilizers against radiolytic degradation, buffers, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

Buffers include acetate buffer, citrate buffer and phosphate buffer.

According to an embodiment, the pharmaceutical composition is an aqueous solution, for example an injectable formulation. According to a particular embodiment, the pharmaceutical composition is a solution for infusion.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ^SHP Handbook on Injectable Drugs, Trissel, 15th ed., pages 622-630 (2009)).

### Methods for Selecting a Subject for GRPR-Antagonist Treatment

The disclosure also relates to methods for determining whether a human patient having tumors can be selected for GRPR-antagonist treatment, said method comprising the steps of:
(i) administering an efficient amount of a radiolabelled GRPR antagonist as a contrast agent for imaging the uptake of said radiolabelled GRPR antagonist,
(ii) acquiring an image by PET/MRI or PET/CT of said patient, and
(iii) comparing with a control image.

The objective of the above method is to select the patient with GRPR-positive tumors, i.e. which patients are good responders to a treatment with a radiolabelled GRPR antagonist. GRPR-positive tumors may be advantageously detected by evaluating the uptake of a radiolabelled GRPR antagonist by PET/MRI or PET/CT imaging after injection of said radiolabelled GRPR antagonist as contrast agent.

As used herein, a good responder is a patient selected from a patient population which shows statistically better response to a treatment as compared to a randomized patient population (i.e. which has not been selected by the selection step of the present method), and/or which shows less side effects to a treatment as compared to a randomized patient population (i.e. which has not been selected by the selection step of the present method).

In one preferred embodiment, a radiolabelled GRPR antagonist for use as contrast agent for imaging the uptake of said radiolabelled GRPR antagonist is a radiolabelled M-NeoB of formula (I):

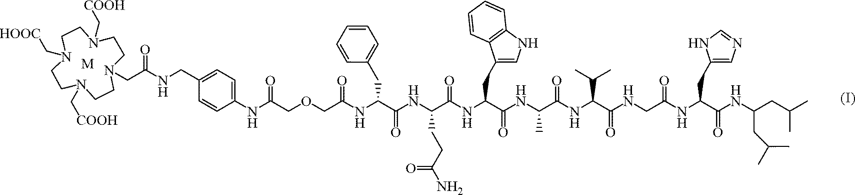

wherein M is a radiometal suitable for PET/MRI or PET/CT imaging. Typically, M is 68-Gallium.

For example, a human patient receives a single dose between 150-250 MBq of [68Ga]-NeoB, typically by intravenous injection.

Images of patient’s body are then acquired by PET/MRI or PET/CT imaging and the images are compared with a control image to identify whether the lesions identified by conventional imaging, for example by MRI, CT, SPECT or PET, are also identified by [68Ga]-GRPR antagonist uptake. Typically, PET/MRI or PET/CT imaging is performed between 1 h and 4 hours after the administration of the radiolabelled GRPR antagonist to the subject, and more preferably with 2 and 3 hours after the administration of the radiolabelled GRPR antagonist to the subject.

In a specific embodiment of the method, said patient is a patient suffering from breast cancer and the radiolabelled GRPR antagonist is a [68Ga]-GRPR antagonist, typically [68Ga]-NeoB.

In a specific embodiment of the method, the patients selected for said treatment are the patients having at least 10%, preferably more than 20%, preferably more than 30%, preferably more than 40%, preferably more than 50%, preferably more than 55%, preferably more than 80%, preferably more than 90%, preferably between 90% and 95% of the lesions detected by conventional imaging which also exhibits [68Ga]-GRPR antagonist uptake as determined by PET/MRI or PET/CT with said [68Ga]-GRPR antagonist.

In specific embodiment, the term “lesion” refers to measurable tumor lesions as defined in the published RECIST document available at http://www.eortc.be. Typically, measurable tumor lesions are lesions with a minimum size (the longest diameter in the plane of measurement is to be recorded) of:
10 mm by CT scan (CT scan slice thickness no greater than 5 mm)
10 mm caliper measurement by clinical exam (lesions which cannot be accurately measured with calipers should be recorded as non-measurable).
20 mm by chest X-ray.

Non-measurable are all other lesions, including small lesions (longest diameter <10 mm or pathological lymph nodes with ≥10 to <15 mm short axis) as well as truly non-measurable lesions. Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

All measurements should be recorded in metric notation, using calipers if clinically assessed. All baseline evaluations should be performed as close as possible to the treatment start.

In specific embodiments, a lesion identified by conventional imaging will be considered a GRPR-positive tumor lesion for the purpose of the present patient selection method, if [68Ga]-NeoB uptake in the lesion is equal or superior (visual assessment) to the spleen uptake.

In other specific embodiments, a lesion is determined as positive for [68Ga]-GRPR antagonist uptake (i.e. GRPR-positive tumor) by determining the ratio between the mean SUV of each region of interest drawn (potential lesions) to the mean SUV of the aorta the ratio between the mean SUV of each region of interest drawn (potential lesions) to the mean SUV of the aorta (SUVr). Typically, a lesion is determined as positive for GRPR overexpression if the SUVr values are above 1.

In particular, the disclosure relates to a pharmaceutical composition of a radiolabeled GRPR-antagonist as described in the previous section, for use as a contrast agent for PET/CT or PET/MRI imaging in determining whether a subject can be selected for a treatment with a radiolabelled GRPR antagonist for treating GRPR-positive tumors, for example GRPR-positive tumors of breast cancer, wherein said subject is selected for the treatment by evaluating uptake of said radiolabelled GRPR antagonist in GRPR-positive tumors by PET/CT or PET/MRI imaging in said subject.

In certain embodiments, the method then further comprises a step of treating GRPR-positive cancer in said patient selected for a treatment by administering a therapeutically efficient amount of a therapeutic agent which comprises the same GRPR antagonist used in step (i) but having a radiometal suitable for therapy.

Typically, the radiolabeled GRPR-antagonist is administered to said subject at a therapeutically efficient amount comprised between 1.85 to 18.5 GBq (50-500 mCi). In specific embodiments, a therapeutically efficient amount of the composition is administered to said subject 1 to 8 times per treatment, for example 2 to 4 times.

Advantageously, the radiolabeled GRPR-antagonist for use as therapeutic agent (treatment step) is labeled with 177-Lu.

In one embodiment, a radiolabelled GRPR antagonist for use as a therapeutic agent is a radiolabelled M-NeoB of formula (I):

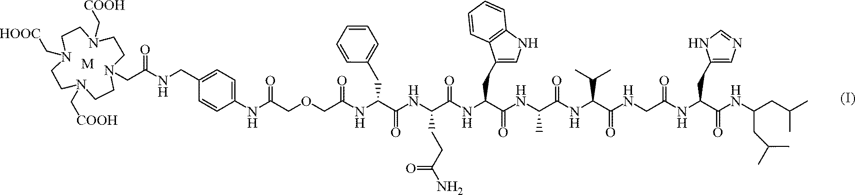

wherein M is a radiometal suitable for therapy. Typically, M is 177-Lutetium.

For example, a patient may be treated with radiolabelled GRPR antagonist, specifically ¹⁷⁷Lu-NeoB, intravenously in 2 to 8 cycles of a 1.85 to 18.5 GBq (50-500 mCi) each.

According to an embodiment, the pharmaceutical composition for use in the treatment step is a solution for infusion, for example comprising 177Lu-NeoB. In particular, it is a solution for infusion of 177Lu-NeoB at 370 MBq/mL.

In certain aspects, the administration of the composition comprising radiolabeled GRPR-antagonist to a subject that has been selected for said treatment can inhibit, delay, and/or reduce tumor growth in the subject. In certain aspects, the growth of the tumor is delayed by at least 50%, 60%, 70% or 80% in comparison to an untreated control subject. In certain aspects, the growth of the tumor is delayed by at least 80% in comparison to an untreated control subject. In certain aspects, the growth of the tumor is delayed by at least 50%, 60%, 70% or 80% in comparison to the predicted growth of the tumor without the treatment. In certain aspects, the growth of the tumor is delayed by at least 80% in comparison to the predicted growth of the tumor without the treatment.

In certain aspects, the administration of the composition comprising radiolabeled GRPR-antagonist to a subject that has been selected for said treatment can increase the length of survival of the subject. In certain aspects, the increase in survival is in comparison to an untreated control subject. In certain aspects, the increase in survival is in comparison to the predicted length of survival of the subject without the treatment. In certain aspects, the length of survival is increased by at least 3 times, 4 times, or 5 times the length in comparison to an untreated control subject. In certain aspects, the length of survival is increased by at least 4 times the length in comparison to an untreated control subject. In certain aspects, the length of survival is increased by at least 3 times, 4 times, or 5 times the length in comparison to the predicted length of survival of the subject without the treatment. In certain aspects, the length of survival is increased by at least 4 times the length in comparison to the predicted length of survival of the subject without the treatment. In certain aspects, the length of survival is increased by at least one week, two weeks, one month, two months, three months, six months, one year, two years, or three years in comparison to an untreated control subject. In certain aspects, the length of survival is increased by at least one month, two months, or three months in comparison to an untreated control subject. In certain aspects, the length of survival is increased by at least one week, two weeks, one month, two months, three months, six months, one year, two years, or three years in comparison to the predicted length of survival of the subject without the treatment. In certain aspects, the length of survival is increased by at least one month, two months, or three months in comparison to the predicted length of survival of the subject without the treatment.

### Radiolabelling Kits of the Disclosure

The present disclosure also relates to a kit comprising
(1) a first vial comprising a GRPR-antagonist as a lyophilized powder, to be reconstituted with a solution of gallium-68 (typically 68GaCl3 in HCl eluted from 68Ge/Ga generator);
(2) a second vial containing the reaction buffer; and,
(3) optionally, instructions for use of the kit,
for use in the pharmaceutical preparation of [68Ga]-labelled peptide (e.g. GRPR-antagonist) to obtain a solution for injection to be used for selecting a patient for a treatment with a radiolabelled Lu-177 GRPR-antagonist.

The kit may be applied in particular for use in the methods as disclosed in the previous sections.

In specific embodiments, the GRPR-antagonist is NeoB as defined above.

### Particular Clauses of the Disclosure Are Presented Hereafter

1. A pharmaceutical composition of a radiolabeled gastrin-releasing peptide receptor (GRPR)-antagonist, for use in treating GRPR-positive tumors in a human subject, wherein said pharmaceutical composition comprises
a radiolabeled GRPR-antagonist of the following formula:

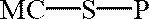

wherein :
   M is a radiometal suitable for therapy, typically 177-Lutetium, and C is a chelator which binds M;
   S is an optional spacer covalently linked between C and the N-terminal of P;
   P is a GRP receptor peptide antagonist covalently bound with its N-terminal to C or to S;
and one or more pharmaceutical excipients,
wherein said subject has been selected for the treatment by positron emitting tomography (PET) / computed tomography (CT) or PET/ magnetic resonance imaging (MRI) with the same GRPR antagonist as defined for the treatment but with ⁶⁸Ga as radiometal for use as contrast agent.

2. The pharmaceutical composition for use according to Item 1, wherein P is of the general formula:

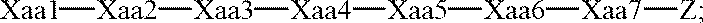

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
all amino acids being independently L- or D- isomer,
Z is selected from —NHOH, —NHNH2, -NH-alkyl, -N(alkyl)2, and -O-alkyl or Z is

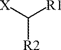

wherein X is NH (forming an amide) or O (forming an ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido or amide substituted aryl or heteroaryl group; and,
one or more pharmaceutically acceptable excipients.

3. The pharmaceutical composition for use according to Item 1 or 2, wherein P is DPhe-Gln-Trp-Ala-Val-Gly-His-NH-CH(CH₂-CH(CH₃)₂)₂.

4. The pharmaceutical composition for use according to any one of Items 1-3 ; wherein the radiolabelled GRPR-antagonist is the compound of formula (I):

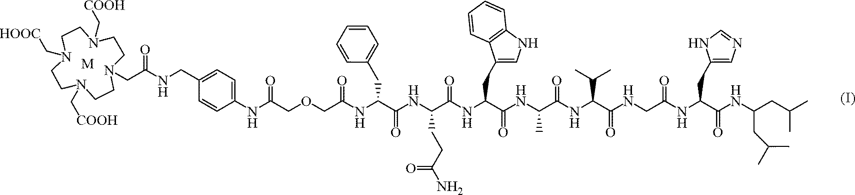

wherein M is a ¹⁷⁷Lu.

5. The pharmaceutical composition according to any of the preceding Items wherein the pharmaceutical composition is an aqueous solution.

6. The pharmaceutical composition according to any of the preceding Items wherein the pharmaceutical composition is a solution for infusion.

7. The pharmaceutical composition for use according to Item 6, wherein a therapeutically efficient dose amount of radiolabeled GRPR-antagonist administered to the subject ranges from 1.85 to 18.5 GBq (50-500 mCi) in 1-8 cycles of infusion.

8. The pharmaceutical composition for use according to any of Items 1 to 7, wherein, a subject has been selected for the treatment by evaluating [⁶⁸Ga]-labeled GRPR antagonist uptake in the lesions as determined by PET/MRI or PET/CT imaging in said subject.

9. The pharmaceutical composition for use according to Item 8, wherein a subject selected for the treatment fulfils at last the following condition: at least 30%, at least 40% or at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging in said subject.

10. The pharmaceutical composition for use according to any of Items 1 to 9, wherein said subject has GRPR-positive solid tumors selected from the group consisting of gastrointestinal stromal tumor (GIST), neuroblastoma, glioblastoma, breast, prostate, lung (small cell and non-small cell), colon-rectum, and renal cancer, preferably breast cancer.

11. The pharmaceutical composition for use according to any of Items 1 to 10, wherein a subject selected for the treatment fulfils at last the following conditions: said subject has breast cancer and at least 30%, at least 40% and preferably at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging.

12. A method for treating cancer in a subject in need thereof, the method comprising administering to said subject a therapeutically efficient amount of a pharmaceutical composition comprising a radiolabeled GRPR-antagonist of the following formula:

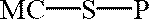

wherein:
M is a radiometal suitable for therapy, for example Lutetium-177 and C is a chelator which binds M; e.g. by forming a complex with M;
S is an optional spacer covalently linked between C and the N-terminal of P;
P is a GRP receptor peptide antagonist, covalently bound with its N-terminal to C or to S and being of the general formula :

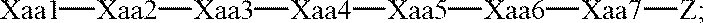

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
Z is selected from —NHOH, —NHNH2, -NH-alkyl, -N(alkyl)2, and -O-alkyl or Z is

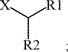

wherein X is NH (forming an amide) or O (forming an ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido or amide substituted aryl or heteroaryl group; wherein said subject has been selected for the treatment by PET/CT or PET/MRI imaging with the same GRPR antagonist as defined for the treatment but with ⁶⁸Ga as radiometal for use as contrast agent.

13. The method of Item 12, wherein P is DPhe-Gln-Trp-Ala-Val-Gly-His-NH-CH(CH₂-CH(CH₃)₂)₂.

14. The method of Item 12 or 13; wherein the radiolabelled GRPR-antagonist is the compound of formula (I):

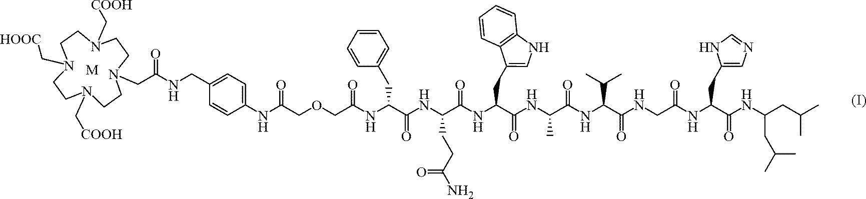

wherein M is a ¹⁷⁷Lu.

15. The method of any one of Items 12-14, wherein the pharmaceutical composition is an aqueous solution.

16. The method of any one of Items 12-15, wherein the pharmaceutical composition is a solution for infusion.

17. The method of any Item 16, wherein a therapeutically efficient dose amount of radiolabeled GRPR-antagonist administered to the patient ranges from 1.85 to 18.5 GBq (50-500 mCi) in 1-8 cycles of infusion.

18. The method of any one of Items 12-17, wherein a subject has been selected for the treatment by evaluating [⁶⁸Ga]-labeled GRPR antagonist uptake in the lesions as determined by PET/MRI or PET/CT imaging in said subject.

19. The method of Item 18, wherein a subject selected for the treatment fulfils at last the following condition: at least at least 30%, at least 40% or at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging in said subject.

20. The method of any one of Items 12-19, wherein said subject has GRPR-positive solid tumors selected among gastrointestinal stromal tumor (GIST), neuroblastoma, glioblastoma, breast, prostate, lung (small cell and non-small cell), colon-rectum, and renal cancer, preferably breast cancer.

21. The method of any one of Items 12-20, wherein a subject selected for the treatment fulfils at last the following conditions: said subject has breast cancer and at least 30%, at least 40% and preferably at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging.

22. A pharmaceutical composition of a radiolabeled GRPR-antagonist, for use as a contrast agent for PET/CT or PET/MRI imaging in determining whether a subject can be selected for a treatment with radiolabelled GRPR-antagonist for treating GRPR-positive tumors, wherein said pharmaceutical composition comprises a radiolabeled GRPR-antagonist of the following formula:

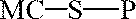

wherein:
M is a radiometal for use as contrast agent in PET imaging, for example 68-Gallium and
C is a chelator which binds M; e.g. by forming a complex with M;
S is an optional spacer covalently linked between C and the N-terminal of P;
P is a GRP receptor peptide antagonist covalently bound with its N-terminal to C or to S, for example of the general formula :

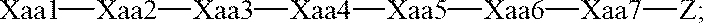

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
All amino acids being, independently, D- or L- isomers,
Z is selected from —NHOH, —NHNH2, -NH-alkyl, -N(alkyl)2, and -O-alkyl or Z is

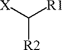

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group.; and, one or more pharmaceutically acceptable excipients, wherein said subject is selected for the treatment by evaluating uptake of said radiolabelled GRPR antagonist in GRPR-positive tumors by PET/CT or PET/MRI imaging in said subject.

23. The pharmaceutical composition for use according to Item 22, wherein P is DPhe-Gln-Trp-Ala-Val-Gly-His-NH-CH(CH₂-CH(CH₃)₂)₂.

24. The pharmaceutical composition for use according to Item 22 or 23 ; wherein the radiolabelled GRPR-antagonist is the compound of formula (I):

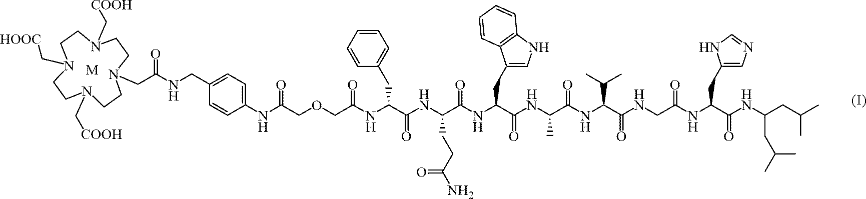

wherein M is a ⁶⁸Ga.

25. The pharmaceutical composition according to Items 22-24 wherein the pharmaceutical composition is an aqueous solution.

26. The pharmaceutical composition according to Items 22-25 wherein the pharmaceutical composition is an injectable solution.

27. The pharmaceutical composition for use according to Item 26, wherein an imaging efficient dose amount of radiolabeled GRPR-antagonist administered to the patient ranges from 150-250 MBq.

28. The pharmaceutical composition for use according to any one of Items 22-27, wherein a subject selected for the treatment fulfils the following condition: at least 30%, at least 40% or at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging in said subject.

29. The pharmaceutical composition for use according to any of Items 22 to 28, wherein said subject has GRPR-positive solid tumors selected among gastrointestinal stromal tumor (GIST), neuroblastoma, glioblastoma, breast, prostate, lung (small cell and non-small cell), colon-rectum, and renal cancer, preferably breast cancer.

30. The pharmaceutical composition for use according to any of Items 22 to 29, wherein a subject selected for the treatment fulfils at last the following conditions: said subject has breast cancer and at least 30%, at least 40% and preferably at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging.

31. A method for determining whether a human patient having tumors can be selected for a treatment with a radiolabelled GRPR antagonist, said method comprising the steps of :
(i) administering an efficient amount of a radiolabelled GRPR antagonist as a contrast agent for imaging the uptake of said radiolabelled GRPR antagonist,
(ii) acquiring an image by PET/MRI or PET/CT of said patient, and
(iii) comparing with a control image.

32. The method of Item 31, further comprising a step of treating GRPR-positive cancer by administering a therapeutically efficient amount of a therapeutic agent which comprises the same GRPR antagonist used in step (i) but having a radiometal suitable for therapy.

33. The method of Item 31 or 32, wherein the radiolabelled GRPR antagonist for use as contrast agent for imaging is a radiolabelled compound of formula (I):

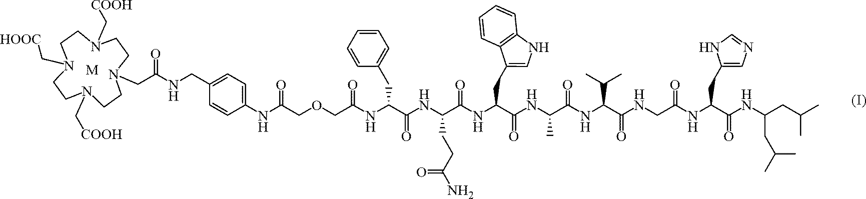

wherein M is 68-Gallium.

34. The method of Item32, wherein the radiometal suitable for therapy is ¹⁷⁷Lu.

35. The method of any one of Items 31-34, wherein the therapeutic agent is administered at least two weeks after step (i).

36. Use of a pharmaceutical composition of a radiolabeled GRPR-antagonist for the manufacture of a contrast agent for PET/CT or PET/MRI imaging in determining whether a subject can be selected for a treatment with radiolabelled GRPR-antagonist for treating GRPR-positive tumors, wherein said pharmaceutical composition comprises a radiolabeled GRPR-antagonist of the following formula:

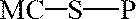

wherein:
M is a radiometal for use as contrast agent in PET imaging, for example 68-Gallium and
C is a chelator which binds M; e.g. by forming a complex with M;
S is an optional spacer covalently linked between C and the N-terminal of P;
P is a GRP receptor peptide antagonist covalently bound with its N-terminal to C or to S, for example of the general formula :

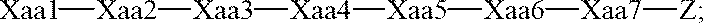

Xaa1 is not present or is selected from the group consisting of amino acid residues Asn, Thr, Phe, 3- (2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) ;
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl )histidine (3-Me)His;
All amino acids being, independently, D- or L- isomers,
Z is selected from —NHOH, —NHNH2, -NH-alkyl, -N(alkyl)2, and -O-alkyl or Z is

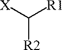

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group.; and, one or more pharmaceutically acceptable excipients, wherein said subject is selected for the treatment by evaluating uptake of said radiolabelled GRPR antagonist in GRPR-positive tumors by PET/CT or PET/MRI imaging in said subject.

37. The use of item 36, wherein P is DPhe-Gln-Trp-Ala-Val-Gly-His-NH-CH(CH₂-CH(CH₃)₂)_{2.}

38. The use of item 36 or 37, wherein the radiolabelled GRPR-antagonist is the compound of formula (I):

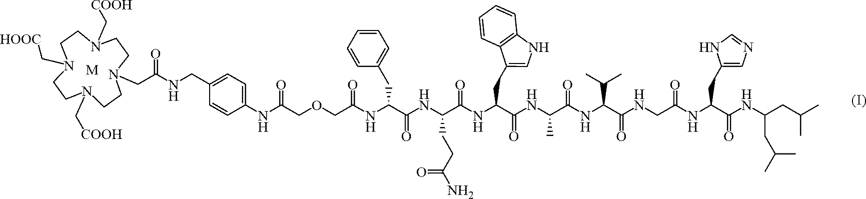

wherein M is a ¹⁷⁷Lu.

39. The use of any one of Items 36 to 38, wherein the pharmaceutical composition is an aqueous solution.

40. The use of any one of Items 36 to 39, wherein the pharmaceutical composition is an injectable solution.

41. The use of Item 40, wherein a therapeutically efficient dose amount of radiolabeled GRPR-antagonist administered to the patient ranges from 150-250 MBq.

42. The use of Item 41, wherein a subject selected for the treatment fulfils at last the following condition: at least 30%, at least 40% or at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging in said subject.

43. The use of Items 36 to 43, wherein said subject has GRPR-positive solid tumors selected among gastrointestinal stromal tumor (GIST), neuroblastoma, glioblastoma, breast, prostate, lung (small cell and non-small cell), colon-rectum, and renal cancer, preferably breast cancer.

44. The use of Items 36 to 44, wherein a subject selected for the treatment fulfils at last the following conditions: said subject has breast cancer and at least 30%, at least 40% and preferably at least 50% of the lesions as detected by conventional imaging in said subject, for example by MRI, CT, SPECT or PET, are also identified by [⁶⁸Ga]-GRPR antagonist uptake as determined by PET/MRI or PET/CT imaging.

### EXAMPLES

### Example 1: Protocol for Treating Human Patients With GRPR-Overexpressing Solid Tumors

In the present clinical protocol, 2 medicinal products are used:
The contrast agent for PET/CT or PET/MRI imaging (Compound 1): [68Ga]-NeoB 50 µg, kit for radiopharmaceutical preparation
The nuclear medicine product (Compound 2): [177Lu]-NeoB 370 MBq/mL (10 mCi/mL), solution for infusion

| | | |
|---|---|---|
| | Pharmaceutical Dosage Form | Route of Administration |
| Compound 1 [68Ga]-NeoB 50 µg | kit for radiopharmaceutical preparation | Intravenous use |
| Compound 2 [177Lu]-NeoB 370 MBq/mL | Solution for infusion | Intravenous use |

### 1.2 Description of Compound 1 - [68Ga]-NeoB, 50 Μg Kit for Radiopharmaceutical Preparation

Compound 1 - [68Ga]-NeoB is a kit for radiopharmaceutical preparation which consists of 2 sterile vials:
Vial 1: NeoB (active ingredient), 50 µg, powder for solution for injection, to be reconstituted with a solution of gallium-68 chloride (68GaCl3) in HCl eluted from a 68Ge/68Ga generator;
Vial 2: Reaction buffer. Vial 2 is to be added to the reconstituted Vial 1.

The kit must be used in combination with a solution of 68Ga in HCl provided by a 68Ge/68Ga generator to obtain [68Ga]-NeoB solution for injection (Radiolabelled Imaging Product) which can be directly injected to the patient.

The volume of [68Ga]-NeoB solution for injection, corresponding to the radioactive dose to be administered, is calculated according to the estimated time of injection, on the basis of the current activity provided by the generator and of physical decay of the radionuclide (half-life = 68 min). The recommended activity to be administered is 3 MBq/Kg (± 10%) (0.08 mCi/Kg), but not more than 250 MBq (6.8 mCi) and not less than 150 MBq (4.1 mCi).

As an example, the composition of the Radiolabelled Imaging Product obtained with the eluate coming from the approved E&Z generator is provided in Table 1.

TABLE 1

Composition of the final injectable solution of [68Ga]-NeoB after reconstitution with 68GaCl3 eluted from an available GMP 68Ge/68Ga generator (E&Z) with a reference activity of 1110 MBq (30 mCi)

| | |
|---|---|
| Component | Quantity |
| Volume and nature of eluate | 5 mL (HCl 0.1N) |
| NeoB | 50 µg |
| [68Ga]-NeoB | ≤ 1,110 MBq (30 mCi) |
| [68Ga]-NeoB | ≤ 0.0177 µg |
| Specific Activity (GBq/Total peptide) | ≤ 34.7 GBq/µmol (938 mCi/µmol) |
| Radioconcentration | ≤ 202 MBq/mL (5.4 mCi/mL) |
| Final Volume obtained | 5.5 mL |

Due to the radioactive nature of the product, a decay of the radionuclide occurs. Consequently, the amount of [68Ga]-NeoB, total radioactivity, specific activity and radioconcentration of the radiolabelled imaging product decreases with time, according with 68Ga half-life. This is a single dose product.

### 1.2 Description of Compound 2: [₁₇₇Lu]-NeoB, 370 MBq/mL (0.1 mCi/mL) Solution for Infusion

Compound 2 is a sterile ready-to-use solution for infusion containing [177Lu]-NeoB as drug substance with a volumetric activity of 370 MBq/mL (10 mCi/mL) at reference date and time (calibration time (tc)). Given the fixed volumetric activity of 370 MBq/mL (10 mCi/mL) at the date and time of calibration, the volume of the solution dispensed varies between 6 mL and 25 mL in order to provide the required amount of radioactivity at the date and time of infusion.

[177Lu]-NeoB is prepared from the NeoB peptide, a 7-mer aminoacid sequence covalently bound to DOTA chelator through the PABZA-DIG linker and [177Lu] chloride. Lutetium (177Lu) has a half-life of 6.647 days. Drug substance synthesis steps are performed in a self-contained closed-system synthesis module which is automated and remotely controlled by GMP compliant software with automated monitoring and recording of the process parameters. Briefly, the manufacturing process consists in the addition of [177Lu] chloride to the reaction vial with corresponding amounts of peptide and reaction buffer. After incubation at selected temperature, the final product is diluted with a formulation solution containing the amount of antioxidant needed for preserving the stability of the radiolabeled product to radiolysis, reaching the volumetric activity of 370 MBq/mL (10 mCi/mL). The final product is sterilized by filtration through a 0.22 µm microbiological filter.

This is a single dose product.

The composition of the [177Lu]-NeoB solution for infusion at the end of production for a dose of 3.70 GBq (100 mCi) (as an example) is shown in Table 2.

Due to the radioactive nature of the product, a natural decay of the radionuclide occurs, which is a property of any radiopharmaceutical, whether it is produced industrially or in-house. As a consequence, the specific activity, total radioactivity, and radio-concentration (volumetric activity) of the Drug Product change over the time.

TABLE 2

Composition of Compound 1[₁₇₇Lu]-NeoB solution for infusion 3.7 GBq (100 mCi)

| | | |
|---|---|---|
| Component | Amount | Function |
| [177Lu]-NeoB | 3.7 GBq (100 mCi) | Active Pharmaceutical Ingredient (API) |
| [177Lu]-NeoB | 8.72 µg | API |
| NeoB | 120 µg | API precursor |
| Specific Activity | 48.6 GBq/µmol (1314 mCi/µmol) | - |
| Radioconcentration (volumetric activity) | 370 MBq/mL (0.1 mCi/mL) | - |
| Final volume | 10 mL | - |

### 1.3 Step 1: Administering an Efficient Amount of NeoB as a Contrast Agent for PET/CT or PET/MRI Imaging

Patients with solid tumors known to overexpress GRPR receive a dose of [68Ga]-NeoB as a contrast agent for imaging as follows:
Regarding radioactivity, a dose between 150 and 250 MBq is considered adequate to ensure appropriate imaging quality throughout the protocol with [68Ga]- as radionuclide.
According to the dosimetry data-set released in the form of interim report from a Phase I clinical study concluded at Innsbruck University in Austria in 6 patients with GIST, effective doses for 68Ga-NeoB ranged from 0.022 mSv/MBq to 0.040 mSv/MBq (mean 0.029 ± 0.06 mSv/MBq), which is in line with the reported effective dose for 68Ga-DOTATATE (0.026 mSv/MBq), a well-established diagnostic tracer (Walker RC, Stabin M, Smith GT, Clanton J, Moore B, Liu E. Measured Human Dosimetry of 68Ga-DOTATATE. Journal of Nuclear Medicine 2013; 54: 855-860). In addition, the mean effective dose for 68Ga-NeoB (0.029 ± 0.06 mSv/MBq) calculated in this clinical study is significantly lower than the calculated human-dose extrapolated from animal model (0.039 mSv/MBq). With a maximum injected activity of 250 MBq (6.8 mCi) the estimated effective dose would be 7.25 mSv per exam, which is lower as the one from the conventional institutional PET imaging.

### 1.4 Step 2: Acquiring Images by PET/CT or PET/MRI Imaging With Compound 1 [68Ga]-NeoB and Selecting Patients for Treatment Step (Step 3)

PET/CT or PET/MRI imaging is performed after the compound 1 administration ideally at 2 h 30 min ±30 min.

The recommended activity to be administered is 3(±10%) MBq/kg (0.08 mCi/kg), but not more than 250 MBq (6.8 mCi) and not less than 150 MBq (4.1 mCi).

All CT scans starts with a topogram (scout) covering from the skull to the mid-thigh. Both CT and PET (axial) field are defined on the topogram. The CT scan is completed using low-dose attenuation correction as per site SOC.

Once the CT acquisition is completed, the gantry moves the subject into the PET positon. The acquisition approximately includes 6-7 PET bed positions, depending on subject’s height. Acquisition times may vary based on the scanners technical capabilities.
3D imaging - each PET bed positon is acquired for 3.5 minutes, providing a total scan time of 21-25 minutes (recommended).

The spleen is the reference region to assess pathological [68Ga]-NeoB uptake. If [68Ga]-NeoB uptake is equal or superior to the spleen one, such uptake is considered specific for overexpression of GRPRs.

A lesion identified by conventional imaging (see next section for the description of conventional imaging) is considered a lesion specific for overexpression of GRPRs for the purpose of the present patient selection method, if [68Ga]-NeoB uptake in the lesion is equal or superior to the spleen uptake as determined by visual assessment.

Should the qualitative visual assessment requires further confirmation, it is suggested to calculate the mean SUVr of each region of interest drawn (potential lesions) to the mean Standardized Uptake as follows :
Value (SUV) of the aorta (better identified on the CT and retrieved on the co-registered PET), as reported below:
SUVr = [SUVmean lesion/SUVmean aorta]

To obtain SUVmean aorta, a spherical volume with a diameter of two (2) cm should be measured within the aortic arch. All SUVr values above one (1) are considered as positive GRPR overexpression.

TABLE 3

CT Equipment Technical Specifications - Reference

| | | |
|---|---|---|
| FEATURE | SPECIFICATION (DIAGNOSTIC HIGH DOSE) | SPECIFICATION (LOW DOSE) |
| kVp | 110-130 | 110-130 |
| mA/mAs | SOC ou | SOC (typically 30% |
| Pitch | 0.8-1.0 | diagnostic dose) 0.8-1.0 |
| Slice Thickness (mm) | 5.0 mm or less, no gap | 5.0 mm or less, no gap |
| Table Feed | 8 mm | 8 mm |
| Topogram | SOC | SOC |

If > 30%, > 40% or preferably >50% of the lesions detected with conventional imaging are identified as well by [68Ga]-NeoB uptake, the patient may be selected for the administration of [177Lu]-NeoB.

### 1.5 Conventional Imaging

### 1.5.1 Diagnostic CT

Routine diagnostic CT scans can be acquired, for example. with or without contrast, as needed, and may cover the chest, abdomen, and pelvis. Additional areas may be scanned as needed.

### 1.5.2 Diagnostic MRI

Routine diagnostic MRI scans, may be completed especially if CT is contraindicated or if the subject presents with a cerebral tumor (e.g. glioblastoma, astrocytoma).

### 1.5.3 PET/CT-Non [68Ga]-NeoB

Routine PET/CT imaging acquired prior to screening and at follow-up time points should be acquired according to institutional SOC. The radiotracer utilized would be based on tumor type by using either [18F]-FDG or [18F]-Choline.

### 1.6 Step 3: Treating With [177Lu]-NeoB Patients Having a [68Ga]-NeoB Tumor Lesion Uptake (as Indicated in the Procedure Described Below) on PET/CT or PET/MRI Scan Performed at step 2

An interval of at least 2 weeks between [68Ga]-NeoB and [177Lu]-NeoB administration is observed.

Patients identified with positive tumor lesions according to [68Ga]-NeoB uptake receives a therapeutic dose amount of 1.85 to 18.5 GBq (50-500 mCi) of [177Lu]-NeoB in 2-8 cycles of infusion.

As per routine procedure in nuclear medicine, a range of ± 10% is accepted for each administered dose without any risk for the safety of the patient.

More specifically, for each single-dose of [177Lu]-NeoB a deviation of ± 10% from the calculated dose is allowed.

[177Lu]-NeoB is administered as a slow infusion. The speed of the infusion does not vary and will be 50 ml/h. Rather, the time of injection increases proportionally to volume and dose. A saline solution is infused in parallel at the same infusion rate (50 ml/h) to flush the tubing. As an example, for a [177Lu]-NeoB dose of 7.40 GBq (200 mCi), depending on the time lapse between the batch production and injection, the estimated volume of infusion could be 25 ml and the duration of infusion is 30 min.

Different infusion methods might be used either pump/flebo infusion methods where the radioactive dose is left in the final vial or syringe infusion methods where the radioactive dose should be withdrawn using a single dose syringe and disposable sterile needle. In all cases, the initial and the residual radioactivity in the vial or in the syringe should be measured by a dose calibrator immediately before and after administration. When the pump method is used, [177Lu]-NeoB is pumped directly into the infusion line. The infusion line is rapidly flushed with at least 25 ml of sodium chloride 9 mg/ml (0.9%) solution for injection after the infusion of [177Lu]-NeoB. When the Flebo infusion method is used, a sodium chloride 9 mg/ml (0.9%) solution for injection gravity flows directly into to the [177Lu]-NeoB solution, which is connected to the infusion line.

The administration of [177Lu]-NeoB is expected to result in a greater effective radiation dose to the target organs (i.e. the disease) compared with the non-specific radiopharmaceutical uptake to the non-target organs. Due to the physical properties of the radionuclide labelling the ligand, the whole-body radiation exposure of patients receiving [177Lu]-NeoB will be high.

The expected benefit of [177Lu]-NeoB relies on the targeted therapeutic delivery of the radioactive payload which will primarily affect the malignant cells, abnormally expressing the GRPR. This principle is known as endo-radiotherapy or peptide receptor radionuclide therapy (PRRT) in the case of NeoB ligand.

### Example 2: Comparison of Number of Lesions Detected by Conventional Imaging Methods and by [68Ga]-NeoB

The primary objective was to characterize preliminary targeting properties of [68Ga] NeoB in patients with malignancies known to overexpress GRPR. The primary efficacy endpoints were:
Number and location of tumor lesions detected by [68Ga]-NeoBOMB1 overall and for each tumor type
Calculation of the ratio tumor/background standard uptake value (SUV) and %injected dose per gram of tissue (ID/g) and calculation of absorbed dose (mGy/MBq) in tumor overall and for each tumor type.

### 1. Number and Location of Tumor Lesions Detected by [68Ga]-NeoB

The number and location of tumor lesions detected by conventional imaging methods and by [68Ga]-NeoB, overall and for each tumor type, are shown respectively in Tables 4 and 5.

TABLE 4

Lesion-Level Analyses of Diagnostics between [68Ga]-NeoB and conventionnal imaging in overall cancer (Breast, Prostate, Colorectal, NSCL and SCL cancers

| Overall cancer N=19 (Breast N=5, Prostate N=5, Colorectal N=5, NSCL N=3 and SCL N=1) | | | Location of Lesion by conventional imaging | | |
|---|---|---|---|---|---|
| | | | Positive | Negative | Total |
| Location of Lesion with [68Ga]-NeoB | Overall | Positive | 87 | 38 | 125 |
| | | Negative | 167 | - | 167 |
| | | Total | 254 | 38 | 292 |
| | Nodal | Positive | 18 | 1 | 19 |
| | | Negative | 36 | - | 36 |
| | | Total | 54 | 1 | 55 |
| | Skeletal | Positive | 17 | 12 | 29 |
| | | Negative | 85 | - | 85 |
| | | Total | 102 | 12 | 114 |
| | Skin/Superficial | Positive | 2 | 0 | 2 |
| | | Negative | 1 | - | 1 |
| | | Total | 3 | 0 | 3 |
| | Soft Tissus/Visceral | Positive | 50 | 25 | 75 |
| | | Negative | 45 | - | 45 |
| | | Total | 95 | 25 | 120 |

In general, the lesions identified by conventional imaging are 254 and among these 254 lesions, 87 are also identified by [68Ga]-NeoB. Therefore 34.3% of the lesions identified by conventional imaging are also identified by [68Ga]-NeoB regardless the cancer type (100% x double positive/total number of lesions identified in conventional imaging). The 2-sided exact binomial confidence interval (CI) is (28,4-40,4).

By going into detail, it in soft tissue and visceral tissue regardless the cancer type, 52.6% of the lesions identified by conventional imaging are also identified by [68Ga]-NeoB.

TABLE 5

Lesion-Level Analyses of Diagnostics between [68Ga]-NeoB and conventionnal imaging in each tumor type

| By tumor type | | | Location of Lesion by conventional imaging | | |
|---|---|---|---|---|---|
| | | | Positive | Negative | Total |
| Location of Lesion with [68Gal]-NeoB | Breast N=5 | Positive | 48 | 37 | 85 |
| | | Negative | 44 | - | 44 |
| | | Total | 92 | 37 | 129 |
| | Prostate N=5 | Positive | 10 | 1 | 11 |
| | | Negative | 59 | - | 59 |
| | | Total | 69 | 1 | 70 |
| | Colorectal N=5 | Positive | 18 | 0 | 18 |
| | | Negative | 43 | - | 43 |
| | | Total | 61 | 0 | 61 |
| | NSCL N=3 | Positive | 10 | 0 | 10 |
| | | Negative | 20 | - | 20 |
| | | Total | 30 | 0 | 30 |
| | SCL N=1 | Positive | 1 | 0 | 1 |
| | | Negative | 1 | - | 1 |
| | | Total | 2 | 0 | 2 |

Specifically, the location of lesion has been measured for each tumor type indenpendently (Breast N=5, Prostate N=5, Colorectal N=5, NSCL N=3 and SCL N=1).

For Breast cancer, the lesions identified by conventional imaging are 92 and among these 92 lesions, 48 are also identified by [68Ga]-NeoB. Therefore 52.2% of the lesions identified by conventional imaging are also identified by [68Ga]-NeoB (100% x double positive/total number of lesions identified in conventional imaging). The 2-sided exact binomial confidence interval (CI) at 95% is (41.5-62.7).

For Prostate cancer, the lesions identified by conventional imaging are 69 and among these 69 lesions, 10 are also identified by [68Ga]-NeoB. Therefore 14.5% of the lesions identified by conventional imaging are also identified by [68Ga]-NeoB (100% x double positive/total number of lesions identified in conventional imaging). The 2-sided exact binomial confidence interval (CI) at 95% is (7.2-25).

For Colorectal cancer, the lesions identified by conventional imaging are 61 and among these 61 lesions, 18 are also identified by [68Ga]-NeoB. Therefore 29.5% of the lesions identified by conventional imaging are also identified by [68Ga]-NeoB (100% x double positive/total number of lesions identified in conventional imaging). The 2-sided exact binomial confidence interval (CI) at 95% is (18.5-42.6).

For NSCL cancer, the lesions identified by conventional imaging are 30 and among these 30 lesions, 10 are also identified by [68Ga]-NeoB. Therefore 33.3% of the lesions identified by conventional imaging are also identified by [68Ga]-NeoB (100% x double positive/total number of lesions identified in conventional imaging). The 2-sided exact binomial confidence interval (CI) at 95% is (17.3-52.8).

For SCL cancer, the lesions identified by conventional imaging are 2 and among these 2 lesions, 1 are also identified by [68Ga]-NeoB. Therefore 50% of the lesions identified by conventional imaging are also identified by [68Ga]-NeoB (100% x double positive/total number of lesions identified in conventional imaging). The 2-sided exact binomial confidence interval (CI) at 95% is (1.3-98.7).

### 2. Ratio Tumor/Background SUV

### Reference Organ (Visual Assessment)

The central reviewer performed a qualitative visual assessment to determine the most appropriate reference organ to serve as visual reference.

In general, 2 different patterns were observed according to their uptake level:
Lesions with low [⁶⁸Ga]-NeoB uptake (representing the majority of patients) showed an uptake similar to that seen in spleen and MBP.
Lesions with high [⁶⁸Ga]-NeoB uptake (2-3 patients) showed an uptake similar to or higher than the liver.

Although muscle, liver, spleen and MBP had almost homogeneous uptake, muscle tissue exhibited an uptake too modest to be considered a reference region. Depending on the intensity used to define positivity for lesions, the threshold could be based on either liver (high) or MBP/spleen (mild/moderate) uptake. Liver could be a suitable region of reference according to the radiotracer characteristics but it is a rather frequent localization for metastases, which might uneven the accuracy of the ratio. The spleen and MBP have similar features regarding radiotracer biodistribution so either one can be used, in particular MBP should be used in case the localization of the spleen is challenging (e.g. small size, accessory spleen, surgery). Reference organs most suitable for SUVr calculation seem to be either spleen or MBP.

### Non-Dosimetry Group

In general, SUVₘₑₐₙ and SUVₘₐₓ values in lesions peaked at 1 h 30 minutes overall.

The highest SUVₘₑₐₙ values were observed for prostatic tumors in soft tissue/visceral (14.043 g/mL) and overall (11.638 g/mL) locations at 1 h 30 minutes.

The highest SUVₘₐₓ values were observed for breast tumors overall (23.120 g/mL) and in soft tissue/visceral (22.140 g/mL) at 2 h 30 minutes. The accumulation of the radio tracer in the lesions of the patients with breast tumor is in agreement with the high expression of GRPR in this type of tumor (Dalm et al, 2015). However it does not imply that imaging time point should be set up at 2 h 30 min for breast cancer patients, as a good signal is already detected at 1 h 30 min. Nonetheless, this feature is an additional argument in favor of potential longer effect of the therapeutic compound that would keep accumulating overtime at lesion level, hence delivering targeted radiations toward therapeutic effect.

Independently of tumor origin, the expression of GRPR drives the retention and accumulation of the tracer in the clusters of cells expressing such receptor.

### Dosimetry Group

The dosimetry group comprised only patients with breast cancer (N=2). In general, SUVₘₑₐₙ values in lesions peaked at 15 minutes for the dosimetry group, reaching the highest values in soft tissue/visceral location at 15 minutes (9.240 g/mL) and 4 h (9.140 g/mL). In general, SUVₘₐₓ values in lesions peaked at 4 h. The highest SUVₘₐₓ values were observed in soft tissue/visceral at 4 h (26.380 g/mL) and at 2 h (25.830 g/mL).

The relative radiotracer uptake (reported in %ID/g) in source organs and tumor lesions per time point for each patient of the dosimetry group is presented in Table 6 and Table 7. The source organ with highest %ID/g was pancreas, followed by urinary bladder and liver at all the time points for both patients. The results are in line with findings published elsewhere. Accordingly, highest relative tracer uptake following administration of a bombesin antagonist is expected in pancreas, kidney, and liver (Roivainen et al, 2013). The highest %ID/g in tumor lesions was measured in spine (T3) for patient FR01-008 and in liverR2 (T5) for patient FR01-009.

TABLE 6

Relative radiotracer uptake in source organs and tumor lesions (%ID/g) per time point for patient FR01-008

| | | | | |
|---|---|---|---|---|
| Source organs and tumor lesion | 15 min¹/38.6 min² | 60 min¹/62.6 min² | 120 min¹/123.2 min² | 240 min¹/221.1 min² |
| Alveolar-interstitial (Lungs) | 2.1842E-03 | 9.4759E-04 | 6.7856E-04 | 6.2082E-04 |
| Heart wall | 6.0002E-03 | 2.4054E-03 | 1.6323E-03 | 1.3018E-03 |
| Kidneys | 6.8821E-03 | 5.7654E-03 | 2.3856E-03 | 1.4872E-03 |
| Liver | 7.9383E-03 | 2.9551E-03 | 1.9746E-03 | 1.6194E-03 |
| Pancreas | 4.7107E-02 | 4.8359E-02 | 5.4453E-02 | 6.2796E-02 |
| Red (active) marrow | 3.3050E-03 | 1.1626E-03 | 9.1618E-04 | 3.4645E-04 |
| Spleen | 4.0928E-03 | 2.1098E-03 | 1.4084E-03 | 1.2014E-03 |
| Urinary bladder wall | 3.1317E-02 | 4.2590E-02 | 2.1412E-02 | 1.7902E-02 |
| T1-Chest | 3.4664E-03 | 2.1832E-03 | 1.4935E-03 | 1.2884E-03 |
| T2-Left rib | 3.8441E-03 | 2.5064E-03 | 1.7251E-03 | 1.6681E-03 |
| T3-Spine | 4.6895E-03 | 2.2489E-03 | 1.8497E-03 | 1.9539E-03 |

¹Time of PET/CT acquisition as per protocol
²Actual time of PET/CT acquisition

TABLE 7

Relative radiotracer uptake in source organs and tumor lesions (%ID/g) per time point for patient FR01-009

| | | | | |
|---|---|---|---|---|
| Source organs and tumor lesion | 15 min¹/17.8 min² | 60 min¹/60.7 min² | 120 min¹/123.9 min² | 240 min¹/207.9 min² |
| Alveolar-interstitial (Lungs) | 1.2389E-03 | 8.8408E-04 | 5.9060E-04 | 3.8616E-04 |
| Heart wall | 3.4039E-03 | 2.0707E-03 | 1.4177E-03 | 9.2790E-04 |
| Kidneys | 4.8041E-03 | 3.8002E-03 | 5.0466E-03 | 2.7828E-03 |
| Liver | 8.6617E-03 | 5.6389E-03 | 4.2840E-03 | 3.1677E-03 |
| Pancreas | 2.1462E-02 | 2.9092E-02 | 3.4501E-02 | 3.7447E-02 |
| Red (active) marrow | 1.8563E-03 | 1.3554E-03 | 9.9765E-04 | 7.2189E-04 |
| Spleen | 3.3766E-03 | 2.5609E-03 | 1.9132E-03 | 1.4477E-03 |
| Urinary bladder wall | 2.6816E-02 | 6.4796E-02 | 4.0547E-02 | 1.1045E-01 |
| T1-lungL | 3.6731E-03 | 4.5478E-03 | 4.8073E-03 | 3.8517E-03 |
| T2-lungR | 4.6570E-03 | 4.6265E-03 | 3.7702E-03 | 2.4820E-03 |
| T3-liverL | 1.3530E-02 | 1.7998E-02 | 9.8384E-03 | 1.7824E-02 |
| T4-liverR1 | 1.3040E-02 | 1.3998E-02 | 1.5267E-02 | 1.1635E-02 |
| T5-liverR2 | 1.5035E-02 | 1.9278E-02 | 2.1971E-02 | 2.1185E-02 |
| T6-sacrumL | 3.1468E-03 | 2.8863E-03 | 2.5397E-03 | 1.8825E-03 |
| T7-liverP | 1.2180E-02 | 1.1801E-02 | 1.2422E-02 | 1.0821E-02 |
| T8-liverR | 1.0793E-02 | 9.6064E-03 | 1.0878E-02 | 1.0121-E02 |

R1 and R2 correspond to the coding assigned in the dosimetry report to distinguish between different tumor lesions observed in the same organ
¹Time of PET/CT acquisition as per protocol
²Actual time of PET/CT acquisition

### SUVr

SUVr is the ratio between SUV mean of each lesion detected by [⁶⁸Ga]-NeoBOMB1 and the SUVmean of the region of reference.

Semi quantitative results are in agreement with the central visual review with a SUVr around 1 corresponding to a mild uptake. Notwithstanding, at patient level, most patients had a SUVr> 1, using spleen or MBP (aorta or heart) as reference organ. The quantitative analysis supports the conclusion from the central reviewer for reference organ based on visual assessment.

In case of areas with moderate [⁶⁸Ga]-NeoBOMB1 uptake, it is suggested to calculate the SUVr, i.e. the ratio of such area to that of the MBP or spleen, and consider such area a lesion in case of ratio above 1.

### Example 3: Exploratory Efficacy Results: Absorbed Tumor Doses for Potential Application of [177Lu]-NeoBOMB1

The activity distributions of [⁶⁸Ga]-NeoBOMB1 over time were used in an exploratory manner to estimate radiation dosimetry of [¹⁷⁷Lu]-NeoBOMB1 by assuming the same biological clearance rate for both compounds.

Absorbed dose extrapolations to target organs are presented in Table 8 for patient FR01-008, and in Table 10 for patient FR01-009. Absorbed dose extrapolations to tumor lesions are presented in Table 9 for patient FR01-008, and in Table 11 for patient FR01-009.

TABLE 8

Absorbed dose extrapolation to target organs for patient FR01-008

| | |
|---|---|
| Target organs | Absorbed dose per MBq (mGy/MBq) |
| Alveolar-interstitial (Lungs) | 5.15E-02 |
| Heart | 3.43E-02 |
| Liver | 6.96E-02 |
| Kidneys | 5.42E-02 |
| Spleen | 4.80E-02 |
| Pancreas | 1.24E+01 |
| Bone marrow | 8.78E-03 |
| Urinary bladder wall | 7.34E-03 |
| Left colon stem cell layer | 6.10E-03 |
| Right colon stem cell layer | 1.24E-02 |
| Small intestine stem cell layer | 3.77E-02 |
| Stomach stem cell layer | 8.18E-02 |
| Endosteal cells | 3.58E-03 |
| Muscle | 2.10E-03 |
| Thyroid | 6.28E-04 |
| Adrenals | 2.93E-02 |
| Prostate | 0.00E+00 |
| Testes or ovary | 1.21E-03 |
| Gallbladder wall | 8.64E-02 |
| Esophagus | 4.03E-03 |
| Thymus | 1.60E-03 |
| Brain | 4.56E-05 |
| Lens of the eye | 1.42E-04 |
| Breasts | 2.00E-03 |
| Skin | 1.08E-03 |
| Uterus | 1.72E-03 |
| Effective whole body dose (mSv/MBq) | 1.39E-01 |

TABLE 9

Absorbed dose extrapolation to tumor lesions for patient FR01-008

| | |
|---|---|
| Tumors | Absorbed dose per MBq (mGy/MBq) |
| T1-Chest | 1.14E-02 |
| T2-Left rib | 4.09E-02 |
| T3-Spine | 2.88E-01 |

TABLE 10

Absorbed dose extrapolation to target organs for patient FR01-009

| | |
|---|---|
| Target organs | Absorbed dose per MBq (mGy/MBq) |
| Alveolar-interstitial (Lungs) | 1.33E-02 |
| Heart | 8.13E-03 |
| Liver | 2.79E-02 |
| Kidneys | 3.08E-02 |
| Spleen | 1.33E-02 |
| Pancreas | 7.32E+00 |
| Bone marrow | 4.62E-03 |
| Urinary bladder wall | 6.11E-03 |
| Left colon stem cell layer | 1.76E-03 |
| Right colon stem cell layer | 3.50E-03 |
| Small intestine stem cell layer | 1.05E-02 |
| Stomach stem cell layer | 2.24E-02 |
| Endosteal cells | 1.72E-03 |
| Muscle | 6.14E-04 |
| Thyroid | 1.80E-04 |
| Adrenals | 8.25E-03 |
| Prostate | 0.00E+00 |
| Testes or ovary | 6.82E-04 |
| Gallbladder wall | 2.38E-02 |
| Esophagus | 1.13E-03 |
| Thymus | 4.47E-04 |
| Brain | 1.79E-05 |
| Lens of the eye | 4.19E-05 |
| Breasts | 5.56E-04 |
| Skin | 3.10E-04 |
| Uterus | 1.13E-03 |
| Effective whole body dose (mSv/MBq) | 7.51E-02 |

TABLE 11

Absorbed dose extrapolation to tumor lesions in patient FR01-009

| Tumors | Absorbed dose per MBq (mGy/MBq) |
|---|---|
| T1-lungL | 1.42 |
| T2-lungR | 0.48 |
| T3-liverL | 3.28 |
| T4-liverR1 | 0.09 |
| T5-liverR2 | 1.34 |
| T6-sacrumL | 0.02 |
| T7-liverP | 0.13 |
| T8-liverR | 1.88 |

R1 and R2 correspond to the coding assigned in the dosimetry report to distinguish between different tumor lesions observed in the same organ

The extrapolation from [⁶⁸Ga]-NeoB to [¹⁷⁷Lu]-NeoB can be challenging and is not considered appropriate, due to the different half-lives ([⁶⁸Ga]-NeoB: 1 h; [¹⁷⁷Lu]-NeoB: 6.6 d). The time activity curves obtained in this study consist of [⁶⁸Ga]-NeoB profiles up to 4 hours only. The time integrated activity coefficients (TIACs) are estimated by integration over these 4 timepoints and an extrapolation after last time point to infinity based on physical decay. This leads to an overestimation of the organ exposure. Nevertheless, this extrapolation was performed with exploratory purposes, in order to have a rough indication of what could be the organ absorbed doses after administration of 177Lu-labelled compound.

The absorbed dose data in Table 8 and in Table 10 show that, as expected, the potential critical target organ is the pancreas. However, the extrapolated absorbed dose in this organ for a possible first in human dose of 1.85 GBq is below the threshold value for pancreas based on external beam radiation. Regarding the absorbed doses in other organs, these are estimated to be far below the recommended thresholds.

1. A method for treating GRPR-positive tumors in a human subject, comprising administering to the subject a pharmaceutical composition of a radiolabeled gastrin-releasing peptide receptor (GRPR)-antagonist, wherein said pharmaceutical composition comprises
a radiolabeled GRPR-antagonist of the following formula: 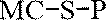
wherein:
M is a radiometal suitable for therapy and C is a chelator which binds M;
S is an optional spacer covalently linked between C and the N-terminal of P;
P is a GRP receptor peptide antagonist covalently bound with its N-terminal to C or to S; and,
one or more pharmaceutically acceptable excipients,
wherein said subject has been selected for the treatment by positron emitting tomography (PET) / computed tomography (CT) or PET/ magnetic resonance imaging (MRI) with the same GRPR antagonist as defined for the treatment but with ⁶⁸Ga as radiometal for use as contrast agent.

2. The method according to claim 1, wherein P is of the general formula: 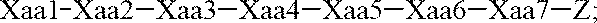
wherein
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
all amino acids being independently L- or D- isomer,
Z is selected from —NHOH, —NHNH2, -NH-alkyl, -N(alkyl)2, and -O-alkyl
or Z is 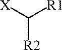
wherein X is NH (forming an amide) or O (forming an ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido or amide substituted aryl or heteroaryl group.

3. The method according to claim 1, wherein P is DPhe-Gln-Trp-Ala-Val-Gly-His-NH-CH(CH₂-CH(CH₃)₂)₂.

4. The method according to claim 1; wherein the radiolabelled GRPR-antagonist is a compound of formula (I): 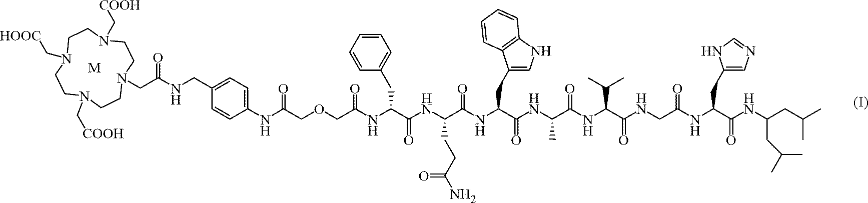
wherein M is ¹⁷⁷Lu.

5. The method according to claim 1 wherein the pharmaceutical composition is a solution for infusion.

6. The method according to claim 1 wherein, the subject has been selected for the treatment by evaluating [⁶⁸Ga]-labeled GRPR antagonist uptake in the lesions as determined by PET/MRI or PET/CT imaging in said subject.

7. The method according to claim 6, wherein the subject selected for the treatment fulfils at least the following condition: at least 50% of the lesions as detected by conventional imaging in said subject are also identified by [⁶⁸Ga]-GRPR antagonist uptake as determined by PET/MRI or PET/CT imaging in said subject.

8. The method according to claim 1, wherein said subject has GRPR-positive solid tumors selected from the group consisting of gastrointestinal stromal tumor (GIST), neuroblastoma, glioblastoma, breast, prostate, lung (small cell and non-small cell), colon-rectum, and renal cancer.

9. A method for treating GRPR-positive tumors in a subject, comprising administering to the subject a pharmaceutical composition of a radiolabeled gastrin-releasing peptide receptor (GRPR)-antagonist, wherein said pharmaceutical composition comprises
a radiolabeled GRPR-antagonist of the following formula: 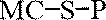
wherein:
M is a radiometal for use as contrast agent in PET imaging;
S is an optional spacer covalently linked between C and the N-terminal of P;
P is a GRP receptor peptide antagonist covalently bound with its N-terminal to C or to S of the general formula:
Xaa1-Xaa2-Xaa3-Xaa4—Xaa5—Xaa6—Xaa7—Z; wherein
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
All amino acids being, independently, D- or L- isomers,
Z is selected from —NHOH, —NHNH2, -NH-alkyl, -N(alkyl)2, and -O-alkyl
or Z is 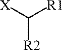
wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group; and,
one or more pharmaceutically acceptable excipients,
wherein said subject is selected for the treatment by evaluating uptake of said radiolabelled GRPR antagonist in GRPR-positive tumors by PET/CT or PET/MRI imaging in said subject.

10. The method according to claim 9, wherein P is DPhe-Gln-Trp-Ala-Val-Gly-His- NH-CH(CH₂-CH(CH₃)₂)₂.

11. The method according to claim 9; wherein the radiolabelled GRPR-antagonist is a compound of formula (I): 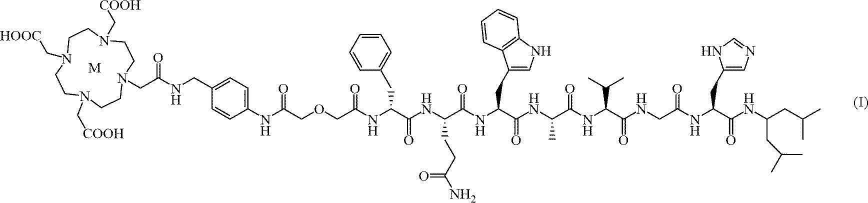
wherein M is ⁶⁸Ga.

12. The method according to claim 9, wherein the subject selected for the treatment fulfils the following condition: at least 30% of the lesions as detected by conventional imaging in said subject are also identified by [⁶⁸Ga]-GRPR antagonist uptake as determined by PET/MRI or PET/CT imaging in said subject.

13. The method according to claim 9, wherein said subject has GRPR-positive solid tumors selected among gastrointestinal stromal tumor (GIST), neuroblastoma, glioblastoma, breast, prostate, lung (small cell and non-small cell), colon-rectum, and renal cancer.

14. A method for selecting a human patient for a treatment with a radiolabelled GRPR antagonist, said method comprising the steps of:
(i) administering an efficient amount of a radiolabelled GRPR antagonist as a contrast agent for imaging the uptake of said radiolabelled GRPR antagonist,
(ii) acquiring an image by PET/MRI or PET/CT of said patient, and
(iii) comparing with a control image.

15. The method of claim 14, further comprising a step of treating GRPR-positive cancer by administering a therapeutically efficient amount of a therapeutic agent which comprises the same GRPR antagonist used in step (i) but having a radiometal suitable for therapy.

16. The method of claim 14, wherein the radiolabelled GRPR antagonist for use as contrast agent for imaging is a compound of formula (I): 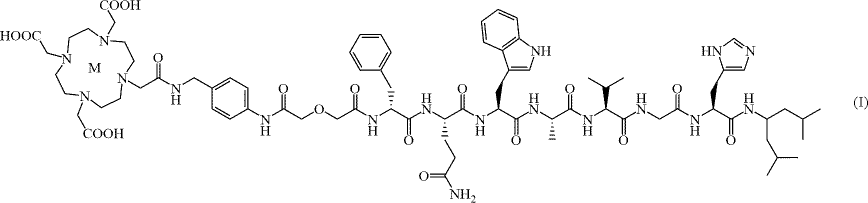
wherein M is 68-Gallium.

17. The method of claim 15, wherein the radiometal suitable for therapy is ¹⁷⁷Lu.

18. The method of claim 15, wherein the therapeutic agent is administered at least two weeks after step (i).
